(19) United States
(12) Patent Application Publication
(10) Pub. No.: US 2005/0235365 A1
(43) Pub. Date: Oct. 20, 2005

(54) Helicokinin-receptor from insects

(76) Inventors: Horst-Peter Antonicek, Berqisch Gladbach (DE); Katrin Schnizler, Rodenbach (DE); Marcus Weidler, Langenfeld (DE)

Correspondence Address: BAYER CROPSCIENCE LP;Patent Department, 100 BAYER ROAD, PITTSBURGH, PA, 15205-9741, US

(21) Appl. No.: 10/511,221

(22) PCT Filed: Apr. 4, 2003
(86) PCT No.: PCT/EP03/03519

§ 371 Date: May 24, 2005

(30) Foreign Application Priority Data
Apr. 16, 2002 (DE) 102 16 738.9

(51) Int. Cl. A01K 67/033; G01N 33/00; G01N 33/53; C07H 21/04; C07K 14/705; C12N 5/06

(52) U.S. Cl. 800/3; 424/405; 800/13; 435/69.1; 435/348; 435/320.1; 530/350; 435/7.2; 530/388.22; 536/23.5

(57) ABSTRACT

The invention relates to polypeptides having the biological activity of a helicokinin receptor, and to polynucleotides which encode the polypeptides, and in particular to their use for finding active compounds for crop protection.

The invention relates to polypeptides having the biological activity of a helicokinin receptor, and to polynucleotides encoding these polypeptides, and in particular to their use for finding active compounds for crop protection.

Traditionally, the development of pesticides has been focused in particular on the chemical and physical properties of the known pesticidally active chemical compounds. As a consequence, the emphasis of further efforts was especially the modification of already existing chemical compounds, and not the finding and development of entirely novel pesticides having new mechanisms of action. Accordingly, it is of particular importance for developing novel pesticides to find novel biological targets (target proteins) from harmful insects for example, to which the potential pesticides can bind and unfold their actions. These novel target proteins can then be expressed in various ways, and the biological function can be examined in various biochemical assays. Furthermore, with the aid of high-throughput assays, the high-throughput screening, it is possible to examine a large number of different chemical substances at relatively low cost and rapidly for their action at the novel target protein. Since, when such a procedure is initiated, it is already clear to which target protein a given chemical substance will bind, it is in particular possible in such a target-orientated approach for the development of a new pesticide, to pay attention to selectivity in the mode of action and thus to safety. Such a chemical compound found by high-throughput screening or in a different way, which has modulating action, for example, on a target protein from harmful insects, can be examined directly for selectivity using homogeneous target protein cloned from one or more mammalian species, to exclude toxic compounds with broad action. Particularly suitable target proteins are those proteins from harmful insects, for example, which do not occur in higher organisms such as mammals.

Particularly suitable target proteins and thus targets for the development of novel insecticides are receptors for biologically active peptides in insects. Peptides regulate most of the important key functions in insects, such as, for example, embryonal and post-embryonal development, ion homeostase, osmoregulation or muscle activity (see Gade et al., 1997a; Osborne, 1996). The biological action of these peptides is mediated by binding to specific receptor proteins in insect cells. Many of these endocrine or neuronal peptides interact with G-protein-coupled receptors (GPCRs: King and Wilson, 1999) which, after binding of a corresponding peptide ligand, activate heterotrimetric G-proteins (Vanden Broeck et al., 1997). Antagonists which are capable of binding to peptide receptors and which may be derived from the natural peptides or else have an entirely novel chemical structure may, for example, interfere with normal insect development, with growth, behaviour or homeostasis, thus generating novel insect-specific and receptor-specific insecticides.

Since the isolation of the neuropeptide Proctolin from preparations from insect muscles (Brown and Starratt, 1975) a large number of peptides from various insect species have been isolated and characterized (see Vanden Broeck et al., 1997; Osborne, 1996, for reviews). Substantial progress has been made in particular in the illustration of the amino acid sequence of such peptides and in the illustration of the biological function in the corresponding insect species. Thus, for example, peptides which regulate the biosynthesis of juvenile hormones (Allatotropins and Allatostatins, Tobe et al., 1994), insulin-like peptides (Lagueux et al., 1990), peptides, which regulate water homeostasis (Coast, 1998), or peptides which can control muscle activity (Holman, 1986; for a review, see Gade, 1997b) have been isolated from various species.

Peptides from the group of the kinins have been isolated from a number of insect species of different families, inter alia from dictyoptera, orthoptera and lepidoptera, (Coast, 1998; Blackburn et al., 1995). They have in common a highly preserved structure including a carboxy-terminal pentapeptide of the sequence phenylalanine-Xaa-Xbb-tryptophane-glycine-amide, where Xaa can be tyrosine, histidine, serine or asparagine and where Xbb can be alanine, but is mainly serine or proline (Coast, 1998). Related peptides have also been isolated from other invertebrates such as molluscs or crabs (Cox et al., 1997; Torfs et al., 1999). The first members of this peptide family were isolated because they are capable of triggering contractions in the isolated intestine in cockroaches (Holman, 1991). Kinines are peptides which are particularly potent in insects and have diuretic action, stimulating the secretion of primary urine in the malpighian corpuscles (Coast, 1998).

The biological functions of the peptides can be examined in various tests in which, for example, muscle activity (Holman, 1991) or the secretion of water and electrolytes (Ramsey, 1954) is measured. Some of these biologically active peptides have been described to cause mortality among the harmful insects if their action is enhanced in vivo by experimental induction (Seinsche et al., 2000).

Whereas a large number of peptides have been isolated, their structure elucidated and the amino acid sequence described, in insects only few receptors are known which are capable of binding endocrine or neuronal peptides. What has been described are, inter alia, receptors for the diuretic hormone from Manduca sexta (Reagan, 1994) or Bombyx mori (Ha et al., 2000), for tachykinin from Drosophila (Li et al., 1991; Monnier et al., 1992), or for galanin from Drosophila (Birgul et al., 1999).

Receptors of kinin peptides have been described in molluscs in the pond snail Lymnea stagnalis (Cox et al., 1997) and in Acari in a tick species (Boophilus microplus, Holmes et al., 2000). In insects, a receptor has only been described in Drosophila melanogaster for a Drosophila kinin (Terhzaz et al., 1999). The cellular response to kinin stimulation in insects is an increase in the intercellular calcium concentration, finally resulting in an influx of chloride ions into the lumen of the malpighian corpuscles (Coast, 1998). In larvae of the harmful insect Heliothis virescens the action of the kinins results, firstly, in an increased fluid secretion in isolated malpighian corpuscles and, secondly, after injection into the hemolymphe of the larvae, in reduction of weight increase and partial mortality (Seinsche et al., 2000). It is therefore of particular interest to provide the receptors for the kinins of harmful insects with economical importance.

Since the genome of Drosophila has been available in part or completely as a sequence searchable in databases, various receptors of this species have been described or predicted (Hauser et al., 1998; Lenz et al., 2000a; Lenz et al., 2000b; Eriksen et al., 2000; Vanden Broeck, 2001; Hewes and Tahert, 2001; WO 00/70980; WO 00/31005). Drosophila melanogaster from the family of the Diptera is an important model organism for insect genetics, but of no major importance as a harmful insect in agriculture. The differences in the amino acid sequences between, for example, homogeneous genes from Drosophila melanogaster and other insects from other families or other invertebrates can be significant and frequently exceeds 50% (Hewes and Taghert, 2001). The differences on the level of the nucleotides are even greater. Therefore, it is frequently not possible, using customary methods of molecular biology (for example by PCR using DNA primers or DNA probes derived, for example, from Drosophila genes), to find and isolate the homologous receptor genes of interest in an invertebrate organism, for example a Lepidoptera species (Pietrantonio et al., 2000).

The present invention is therefore based on the object of providing further receptors to which endocrine or neuronal peptides from insects, in particular from harmful insects of economic importance, can bind and which, via this binding, are capable of mediating the biological functions of these peptides, and of providing assay systems based thereon with a high throughput of test compounds (High Throughput Screening Assays; HTS-Assays).

This object is achieved by providing polypeptides having at least one biological activity of a helicokinin receptor and comprising an amino acid sequence having at least 70% identity, preferably at least 80% identity, particularly preferably at least 90% identity, very particularly preferably at least 95% identity, with the sequence of SEQ ID NO: 2 over a length of at least 20, preferably at least 25, particularly preferably at least 30 consecutive amino acids, and very particularly preferably over their full length.

The degree of identity of the amino acid sequences is preferably determined using the program GAP from the program package GCG, Version 9.1, with standard settings (Devereux et al., 1984).

The term “polypeptides” as used in the present context not only relates to short amino acid chains which are usually referred to as peptides, oligopeptides or oligomers, but also to longer amino acid chains which are usually referred to as proteins. It encompasses amino acid chains which can be modified either by natural processes, such as post-translational processing, or by chemical prior art methods. Such modifications may occur at various sites and repeatedly in a polypeptide, such as, for example, on the peptide backbone, on the amino acid side chain, on the amino and/or the carboxyl terminus. For example, they encompass acetylations, acylations, ADP-ribosylations, amidations, covalent linkages to flavins, haem-moieties, nucleotides or nucleotide derivatives, lipids or lipid derivatives or phosphatidylinositol, cyclizations, disulphide bridge formations, demethylations, cystine formations, formylations, gamma-carboxylations, glycosylations, hydroxylations, iodinations, methylations, myristoylations, oxidations, proteolytic processings, phosphorylations, selenoylations and tRNA-mediated amino acid additions.

The polypeptides according to the invention may exist in the form of “mature” proteins or parts of larger proteins, for example as fusion proteins. They can furthermore exhibit secretion or leader sequences, pro-sequences, sequences which allow simple purification, such as multiple histidine residues, or additional stabilizing amino acids.

The polypeptides according to the invention need not constitute complete receptors, but may also be fragments thereof, as long as they still have at least one biological activity of a complete helicokinin receptor. Polypeptides which, compared to the helicokinin receptor consisting of the polypeptide according to the invention having an amino acid sequence of SEQ ID NO: 2, have an activity which is increased or reduced by 50%, are still considered to be in accordance with the invention.

In comparison to the corresponding region of naturally occurring receptors, the polypeptides according to the invention can have deletions or amino acid substitutions, as long as they still exert at least one biological activity of the complete helicokinin receptors. Conservative substitutions are preferred. Such conservative substitutions comprise variations in which one amino acid is replaced by another amino acid from the following group:
1. small aliphatic residues, non-polar or of little polarity: Ala, Ser, Thr, Pro and Gly;
2. polar negatively charged residues and their amides: Asp, Asn, Glu and Gln;
3. polar positively charged residues: His, Arg and Lys;
4. large aliphatic non-polar residues: Met, Leu, Ile, Val and Cys; and
5. aromatic residues: Phe, Tyr and Trp.

Preferred conservative substitutions are shown in the list below:

| Original | |
|---|---|
| residue | Substitution |
| Ala | Gly, Ser |
| Arg | Lys |
| Asn | Gln, His |
| Asp | Glu |
| Cys | Ser |
| Gln | Asn |
| Glu | Asp |
| Gly | Ala, Pro |
| His | Asn, Gln |
| Ile | Leu, Val |
| Leu | Ile, Val |
| Lys | Arg, Gln, Glu |
| Met | Leu, Tyr, Ile |
| Phe | Met, Leu, Tyr |
| Ser | Thr |
| Thr | Ser |
| Trp | Tyr |
| Tyr | Trp, Phe |
| Val | Ile, Leu |

The term “biological activity of a helicokinin receptor” as used in the present context means binding of a peptide to the peptide receptor.

Preferred embodiments of the polypeptides according to the invention are helicokinin receptors of Lepidoptera, in particular Heliothis virescens.

A particular preferred embodiment of the polypeptides according to the invention is the helicokinin receptor of Heliothis virescens having the amino acid sequence of SEQ ID NO: 2.

The present invention also provides polynucleotides which encode the polypeptides according to the invention.

The polynucleotide according to the invention are, in particular, single-stranded or double-stranded deoxyribonucleic acids (DNA) or ribonucleic acids (RNA). Preferred embodiments are fragments of genomic DNA, which can contain introns, and cDNAs.

A preferred embodiment of the polynucleotides according to the invention is cDNA having the polynucleotide sequence of SEQ ID NO: 1.

Polynucleotides which hybridize under stringent conditions with sequences of SEQ ID NO: 1 are likewise included in the present invention.

The term “to hybridize” as used in the present context describes the process during which a single-stranded nucleic acid molecule undergoes base pairing with a complementary strand. Starting from the sequence information disclosed herein, this allows, for example, DNA fragments to be isolated from insects other than Heliothis virescens which encode polypeptides with the biological activity of helicokinin receptors.

Preferred hybridization conditions are given below:

Hybridization solution: 6×SSC/0% formamide, preferred hybridization solution: 6×SSC/25% formamide.

Hybridization temperature: 34° C., preferred hybridization temperature: 42° C.
Wash step 1: 2×SSC at 40° C.,
Wash step 2: 2×SSC at 45° C.; preferred wash step 2: 0.6×SSC at 55° C.; particularly preferred wash step 2: 0.3×SSC at 65° C.

The present invention furthermore encompasses polynucleotides which have at least 70% identity, preferably at least 80% identity, particularly preferably at least 90% identity, very particularly preferably at least 95% identity, with the sequence of SEQ ID NO: 1 over a length of at least 20, preferably at least 25, particularly preferably at least 30, consecutive nucleotides, and very particularly preferably over their full length.

The degree of identity of the polynucleotide sequences is preferably determined with the aid of the program GAP from the program package GCG, Version 9.1, using standard settings.

The present invention furthermore provides DNA constructs which comprise a polynucleotide according to the invention and a heterologous promoter.

The term “heterologous promoter” as used in the present context refers to a promoter which has properties which differ from the properties of the promoter which controls the expression of the gene in question in the original organism. The term “promoter” as used in the present context generally refers to expression control sequences.

The choice of heterologous promoters depends on whether pro- or eukaryotic cells or cell-free systems are used for expression. Examples of heterologous promoters are the early or late promoter of SV40, of the adenovirus or of the cytomegalovirus, the lac system, the trp system, the main operator and promoter regions of the lambda phage, the fd coat protein control regions, the 3-phosphoglycerate kinase promoter, the acid phosphatase promoter and the yeast α-mating factor promoter.

The invention furthermore provides vectors which contain a polynucleotide according to the invention or a DNA construct according to the invention. All plasmids, phasmids, cosmids, YACs or synthetic chromosomes used in molecular biology laboratories can be used as vectors.

The present invention also provides host cells which contain a polynucleotide according to the invention, a DNA construct according to the invention or a vector according to the invention.

The term “host cell” as used in the present context refers to cells which do not naturally comprise the polynucleotides according to the invention.

Suitable host cells are both prokaryotic cells, such as bacteria from the genera Bacillus, Pseudomonas, Streptomyces, Streptococcus, Staphylococcus, preferably E. coli, and eukaryotic cells, such as yeasts, mammalian cells, amphibian cells, insect cells or plant cells. Preferred eukaryotic host cells are HEK-293, Schneider S2, Spodoptera Sf9, Kc, CHO, COS1, COS7, HeLa, C127, 3T3 or BHK cells and, in particular, Xenopus oocytes.

The invention furthermore provides antibodies which bind specifically to the above-mentioned polypeptides or receptors. Such antibodies are produced in the customary manner. For example, such antibodies may be produced by injecting a substantially immunocompetent host with such an amount of a polypeptide according to the invention or a fragment thereof which is effective for antibody production, and subsequently obtaining this antibody. Furthermore, an immortalized cell line which produces monoclonal antibodies may be obtained in a manner known per se. If appropriate, the antibodies may be labelled with a detection reagent. Preferred examples of such a detection reagent are enzymes, radiolabelled elements, fluorescent chemicals or biotin. Instead of the complete antibody, it is also possible to employ fragments which have the desired specific binding properties. The term “antibodies” as used in the present context therefore also extends to parts of complete antibodies, such as Fa, F(ab′)₂ or Fv fragments, which are still capable of binding to the epitopes of the polypeptides according to the invention.

The polynucleotides according to the invention can be used, in particular, for generating transgenic invertebrates. These may be employed in assay systems which are based on an expression, of the polypeptides according to the invention, which deviates from the wild type. Based on the information disclosed herein, it is furthermore possible to generate transgenic invertebrates where expression of the polypeptides according to the invention is altered owing to the modification of other genes or promoters.

The transgenic invertebrates are generated, for example, in the case of Drosophila melanogaster, by P-element-mediated gene transfer (Hay et al., 1997) or, in Caenorhabditis elegans, by transposon-mediated gene transfer (for example by Tcl; Plasterk, 1996).

The invention therefore also provides transgenic invertebrates which contain at least one of the polynucleotides according to the invention, preferably transgenic invertebrates of the species Drosophila melanogaster or Caenorhabditis elegans, and their transgenic progeny. The transgenic invertebrates preferably contain the polypeptides according to the invention in a form which deviates from the wild type.

The present invention furthermore provides methods of preparing the polypeptides according to the invention. To prepare the polypeptides encoded by the polynucleotides according to the invention, host cells which contain a polynucleotide according to the invention can be cultured under suitable conditions, where the polynucleotide to be expressed may be adapted to the codon usage of the host cells. Thereupon, the desired polypeptides can be isolated from the cells or the culture medium in a customary manner. The polypeptides may also be produced in in vitro systems.

A rapid method of isolating the polypeptides according to the invention which are synthesized by host cells using a polynucleotide according to the invention starts with the expression of a fusion protein, it being possible for the fusion partner to be affinity-purified in a simple manner. For example, the fusion partner may be glutathione S-transferase. The fusion protein can then be purified on a glutathione affinity column. The fusion partner can then be removed by partial proteolytic cleavage, for example at linkers between the fusion partner and the polypeptide according to the invention to be purified. The linker can be designed such that it includes target amino acids, such as arginine and lysine residues, which define sites for trypsin cleavage. To generate such linkers, standard cloning methods using oligonucleotides may be employed.

Other purification methods which are possible are based on preparative electrophoresis, FPLC, HPLC (for example using gel filtration, reversed-phase or moderately hydrophobic columns), gel filtration, differential precipitation, ion-exchange chromatography and affinity chromatography.

Since the helicokinin receptors according to the invention constitute membrane proteins, the purification methods preferably involve detergent extractions, for example using detergents which have no, or little, effect on the secondary and tertiary structures of the polypeptides, such as nonionic detergents.

The purification of the polypeptides according to the invention can encompass the isolation of membranes, starting from host cells which express the polynucleotides according to the invention. Such cells preferably express the polypeptides according to the invention in a sufficiently high copy number, so that the polypeptide quantity in a membrane fraction is at least 10 times higher than that in comparable membranes of cells which naturally express the receptors; particularly preferably, the quantity is at least 100 times, very particularly preferably at least 1 000 times, higher.

The terms “isolation or purification” as used in the present context mean that the polypeptides according to the invention are separated from other proteins or other macromolecules of the cell or of the tissue. The protein content of a composition containing the polypeptides according to the invention is preferably at least 10 times, particularly preferably at least 100 times, higher than in a crude host cell extract. The purity or the protein content of the preparations can be determined in a manner known per se, for example by SDS-polyacrylamide gel electrophoreses.

The polypeptides according to the invention may also be affinity-purified without a fusion partner with the aid of antibodies which bind to the polypeptides.

The present invention furthermore provides methods for preparing the polynucleotides according to the invention. The polynucleotides according to the invention can be prepared in a customary manner. For example, all of the polynucleotides can be synthesized chemically, or else only short sections of the polynucleotides according to the invention can be synthesized chemically, and such oligonucleotides can be radiolabelled or labelled with a fluorescent dye. The labelled oligonucleotides can be used for screening cDNA libraries generated starting from insect mRNA or for screening genomic libraries generated starting from insect genomic DNA. Clones which hybridize with the labelled oligonucleotides are chosen for isolating the DNA in question. After characterization of the isolated DNA, the polynucleotides according to the invention are obtained in a simple manner.

Alternatively, the polynucleotides according to the invention can also be prepared by means of PCR methods using chemically synthesized oligonucleotides.

The term “oligonucleotide(s)” as used in the present context denotes DNA molecules composed of 10 to 50 nucleotides, preferably 15 to 30 nucleotides. They are synthesized chemically and can be used as probes.

The polynucleotides or polypeptides according to the invention allow novel active compounds for crop protection and/or pharmaceutically active compounds for the treatment of humans and animals to be identified, such as chemical compounds which, being modulators, in particular agonists or antagonists, alter the properties of the helicokinin receptors according to the invention. To this end, a recombinant DNA molecule comprising at least one polynucleotide according to the invention is introduced into a suitable host cell. The host cell is grown in the presence of a compound or a probe comprising a variety of compounds under conditions which allow expression of the receptors according to the invention. A change in the receptor properties can be detected, for example, as described below in Example 2. This allows, for example, insecticidal substances to be found.

Receptors alter the concentration of intracellular cAMP or intracellular calcium via interaction with G-proteins, preferably after previously having been activated. Thus, changes in the receptor properties by chemical compounds can be measured after heterologous expression, for example by measuring the intracellular cAMP concentrations directly via ELISA assay systems (Biomol, Hamburg, Germany) or RIA assay systems (NEN, Schwalbach, Germany) in HTS format. An indirect measurement of the cAMP concentration is possible with the aid of reporter genes (for example luciferase), whose expression depends on the cAMP concentration (Stratowa et al., 1995). The coexpression of receptors with specific G-proteins, for example Gα15, Gα16 or else chimeric G-proteins, in heterologous systems and measuring the increase in calcium, for example using fluorescent dyes or equorin, is an alternative possibility of carrying out the screening (Stables et al., 1997, Conklin et al., 1993).

Furthermore, the binding of GTP to the activated G-protein can be used as a read-out system for assaying substances.

The polynucleotides or polypeptides according to the invention also allow the detection of compounds which bind to the receptors according to the invention, without it being necessary to measure a change of activity of the receptors. For example, host cells containing the polynucleotides according to the invention and expressing the corresponding receptors or polypeptides or the gene products themselves are brought into contact with a compound or a mixture of compounds under conditions allowing the interaction of at least one compound with the host cells, the receptors or the individual polypeptides. In such binding experiments, the polypeptides according to the invention can be employed in labelled form.

The term “agonist” as used in the present context refers to a molecule which activates the receptors according to the invention.

The term “antagonist” as used in the present context refers to a molecule which displaces an agonist from its binding site.

The term “modulator” as used in the present context constitutes the generic term for agonist and antagonist. Modulators can be small organochemical molecules, peptides or antibodies which bind to the polypeptides according to the invention. Other modulators may be small organochemical molecules, peptides or antibodies which bind to a molecule which, in turn, binds to the polypeptides according to the invention, thus affecting their biological activity. Modulators may constitute mimetics of natural substrates and ligands.

The modulators are preferably small organochemical compounds.

The binding of the modulators to the polypeptides according to the invention can alter the cellular processes in a manner which leads to the death, to paralysis or to sterility of the insects treated therewith. In vivo tests on insects, insect larvae or insect eggs to verify the insecticidal properties of the modulators found are generally known.

The present invention therefore also extends to the use of modulators of the polypeptides according to the invention as insecticides or pharmaceuticals—hereinbelow referred to as ‘active compounds’.

The active compounds can be converted to the customary formulations, such as solutions, emulsions, wettable powders, suspensions, powders, dusting agents, pastes, soluble powders, granules, suspo-emulsion concentrates, natural and synthetic materials impregnated with active compound and very fine capsules in polymeric substances.

These formulations are produced in a known manner, for example by mixing the active compounds with extenders, that is liquid solvents and/or solid carriers, if appropriate with the use of surfactants, that is emulsifiers and/or dispersants and/or foam-formers.

It the extender used is water, it is also possible to use organic solvents, for example, as auxiliary solvents. Essentially, the following are suitable liquid solvents: aromatics, such as xylene, toluene or alkylnaphthalenes, chlorinated aromatics and chlorinated aliphatic hydrocarbons, such as chlorobenzenes, chloroethylenes or methylene chloride, aliphatic hydrocarbons, such as cyclohexane or paraffins, for example mineral oil fractions, mineral and vegetable oils, alcohols, such as butanol or glycol and their ethers and esters, ketones, such as acetone, methyl ethyl ketone, methyl isobutyl ketone or cyclohexanone, strongly polar solvents, such as dimethylformamide and dimethyl sulphoxide, and water.

Suitable solid carriers are:

for example ammonium salts and ground natural minerals, such as kaolins, clays, talc, chalk, quartz, attapulgite, montmorillonite or diatomaceous earth, and ground synthetic minerals, such as finely divided silica, alumina and silicates; suitable solid carriers for granules are: for example crushed and fractionated natural rocks such as calcite, marble, pumice, sepiolite and dolomite, as well as synthetic granules of inorganic and organic meals, and granules of organic material such as sawdust, coconut shells, maize cobs and tobacco stalks; suitable emulsifiers and/or foam-formers are: for example nonionic and anionic emulsifiers, such as polyoxyethylene fatty acid esters, polyoxyethylene fatty alcohol ethers, for example alkylaryl polyglycol ethers, alkylsulphonates, alkyl sulphates, arylsulphonates and protein hydrolysates; suitable dispersants are: for example lignosulphite waste liquors and methylcellulose.

Tackifiers such as carboxymethylcellulose and natural and synthetic polymers in the form of powders, granules or latices, such as gum arabic, polyvinyl alcohol and polyvinyl acetate, as well as natural phospholipids, such as cephalins and lecithins, and synthetic phospholipids, can be used in the formulations. Other additives can be mineral and vegetable oils.

It is possible to use colourants such as inorganic pigments, for example iron oxide, titanium oxide and Prussian Blue, and organic dyestuffs, such as alizarin dyestuffs, azo dyestuffs and metal phthalocyanine dyestuffs, and trace nutrients such as salts of iron, manganese, boron, copper, cobalt, molybdenum and zinc.

The formulations in general contain between 0.1 and 95% by weight of active compound, preferably between 0.5 and 90%.

The active compounds are preferably employed as crop protection agents, in particular for controlling insects from the order of the Lepidoptera, and for example, Pectinophora gossypiella, Bupalus piniarius, Cheimatobia brumata, Lithocolletis blancardella, Hyponomeuta padella, Plutella xylostella, Malacosoma neustria, Euproctis chrysorrhoea, Lymantria spp., Bucculatrix thurberiella, Phyllocnistis citrella, Agrotis spp., Euxoa spp., Feltia spp., Earias insulana, Heliothis spp., Mamestra brassicae, Panolis flammea, Spodoptera spp., Trichoplusia ni, Carpocapsa pomonella, Pieris spp., Chilo spp., Pyrausta nubilalis, Ephestia kuehniella, Galleria mellonella, Tineola bisselliella, Tinea pellionella, Hofmannophila pseudospretella, Cacoecia podana, Capua reticulana, Choristoneura fumiferana, Clysia ambiguella, Homona magnanima, Tortrix viridana, Cnaphalocerus spp., Oulema oryzae.

The treatment of the plants and parts of plants with the active compounds is carried out directly or by action on their environment, habitat or storage area according to customary treatment methods, for example, by dipping, spraying, evaporating, atomising, broadcasting, brushing on and, in the case of propagation material, in particular in the case of seeds, furthermore by one or multilayer coating.

The active compounds are also suitable for controlling insects which attack agricultural livestock, such as, for example, cattle, sheep, goats, horses, pigs, donkeys, camels, buffalo, rabbits, chickens, turkeys, ducks, geese, honey bees, other domestic animals, such as, for example, dogs, cats, caged birds, aquarium fish, and so-called experimental animals, such as, for example, hamsters, guinea pigs, rats and mice. By controlling these insects, it is intended to reduce mortality and decreased performances (in meat, milk, wool, hides, eggs, honey and the like), so that more economical and simpler animal keeping is possible by using the active compounds.

In the veterinary sector, the active compounds are used in a known manner by enteral administration, for example in the form of tablets, capsules, drinks, drenches, granules, pastes, boluses, the feed-through method, suppositories, by parenteral administration, such as, for example, by means of injections (intramuscular, subcutaneous, intravenous, intraperitoneal and the like), implants, by nasal administration, by dermal administration, for example in the form of dipping or bathing, spraying, pouring-on and spotting-on, washing, dusting, and with the aid of shaped articles which comprise active compound, such as collars, ear tags, tail marks, limb bands, halters, marking devices and the like.

When administered to livestock, poultry, domestic animals and the like, the active compounds can be used as formulations (for example powders, emulsions, flowables) which comprise the active compounds in an amount of 1 to 80% by weight, directly or after dilution by a factor of 100 to 10 000, or they may be used in the form of a chemical bath.

Through the use of host cells or transgenic invertebrates containing the polynucleotides according to the invention it is also possible to detect substances which alter the expression of the receptors.

The above-described polynucleotides according to the invention, vectors and regulatory regions can furthermore be used for finding genes which encode polypeptides which participate in the synthesis, in insects, of functionally similar receptors. Functionally similar receptors are to be understood as meaning in accordance with the present invention receptors which comprise polypeptides which, while differing from the amino acid sequence of the polypeptides described herein, essentially have the same functions.

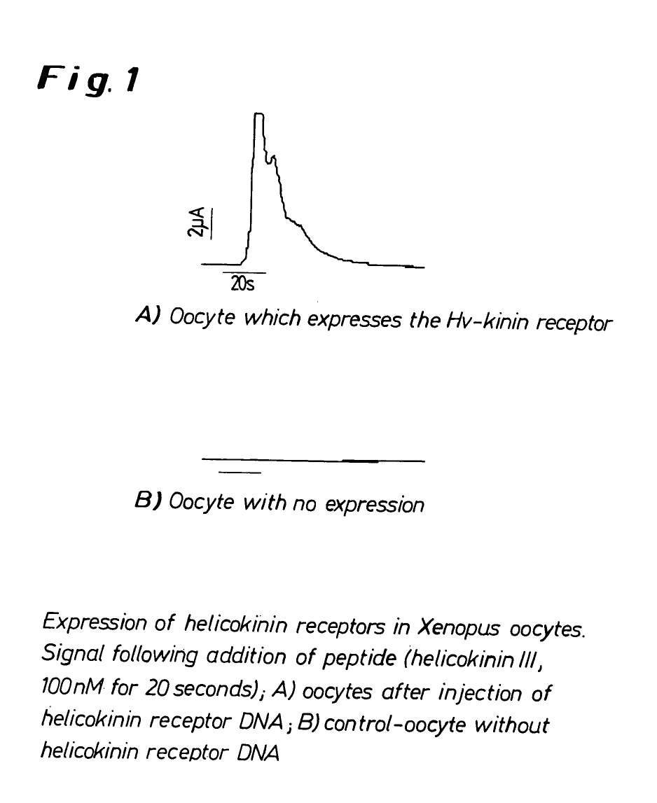

Information on the Sequence Listing and on FIG. 1:

SEQ ID NO: 1 shows the polynucleotide sequence of the isolated helicokinin receptor cDNA. SEQ ID NO: 2 shows the amino acid sequence of the polypeptide encoded by the polynucleotide sequence of SEQ ID NO: 1.

FIG. 1 shows the result of the electrophysiological measure following injection of helicokinin receptor DNA into Xenopus oocytes and the addition of helicokinin peptide (helicokinin III, 100 nM, Blackburn et al., 1995), compared to the application of peptide to control-oocytes into which no helicokinin receptor NDA was injected beforehand.

### EXAMPLES

### Example 1

Isolation of the Above-Described Polynucleotide Sequences

Polynucleotides were manipulated by standard methods of recombinant DNA technology (Sambrook et al., 1989). Nucleotide and amino acid sequences were bioinformatively processed using the program package GCG Version 9.1 (GCG Genetics Computer Group, Inc., Madison Wis., USA).

### Example 2

Generation of the Expression Constructs

Using polymerized chain reaction (PCR), the sequence region of SEQ ID NO: 1 was amplified and cloned into the vector pCMV Script EX (Stratagene, La Jolla, USA).

Heterologous Expression

The helicokinin receptor from Heliothis virescens was functionally expressed in Xenopus oocytes. To this end, some G-protein-activated potassium channels (GIRK1 and GIRK4) are coexpressed in order to measure activation of the receptors (White et al., 1998).

Oocyte Measurements

1. Preparation

The oocytes are obtained from an adult female Xenopus laevis frog (Horst Kähler, Hamburg, Germany). The frogs are kept in large tanks with circulating water at a water temperature of 18-20° C. Parts of the frog's ovary are removed through a small incision in the abdomen (ca. 1 cm), with full anaesthesia. The ovary is then treated for approximately 140 min with 25 ml of collagenase (Type I, C-0130, SIGMA-ALDRICH CHEMIE GmbH, Deisenhofen, Germany; 355 U/ml, prepared with Barth's solution without calcium in mM: NaCl 88, KCl 1, MgSO4 0.82, NaHCO3 2.4, Tris/HCl 5, pH 7.4) with constant shaking. Then, the oocytes are washed with Barth's solution without calcium. Only oocytes at maturity stage V (Dumont, 1972) are selected for further treatment and transferred into microtitre plates (Nunc MicroWell™ Plates, Cat. No. 245128+263339 (Lid), Nunc GmbH & Co. KG, Wiesbaden, Germany), filled with Barth's solution (in mM: NaCl 88, KCl 1, MgSO₄ 0.82, Ca(NO₃)₂ 0.33, CaCl₂ 0.41, NaHCO3 2.4, Tris/HCl 5, pH 7.4) and gentamicin (gentamicin sulfate, G-3632, SIGMA-ALDRICH CHEMIE GmbH, Deisenhofen, Germany; 100 U/ml). The oocytes are then kept in a cooling incubator (Type KB 53, WTB Binder Labortechnik GmbH, Tuttlingen, Germany) at 19.2° C.

2. Injecting the Oocytes

Injection electrodes of a diameter of 10-15 μm are prepared using a Pipette-drawing device (Type L/M-3P-A, List-electronic, Darmstadt-Eberstadt, Germany). Prior to injection, aliquots with the receptor DNA or GIRK1/4-DNA are defrosted and diluted with water to a final concentration of 10 ng/μl. The DNA samples are centrifuged for 120 s at 3 200 g (Type Biofuge 13, Heraeus Instruments GmbH, Hanau, Germany). An extended PE tube is subsequently used as transfer tube to fill the pipettes from the rear end. The injection electrodes are attached to an X, Y, Z positioning system (Treatment Centre EP1090, isel-automation, Eiterfeld, Germany). With the aid of a Macintosh Computer, the oocytes in the microtitre plate wells are approached, and approximately 50 nl of the DNA solution are injected into the oocytes by brief application of pressure (0.5-3 bar, 3-6 s).

3. Electrophysiological Measurements

A 2-electrode voltage clamp with a TURBO TEC-10CD (npi electronic GmbH, Tamm, Germany) amplifier is used to carry out the electro-physiological measurements. The micro pipettes required for this purpose are drawn in two movements from aluminium silicate glass (Capillary tube, Art. No. 14 630 29, 1=100 mm, Ø_{ext.}=1.60 mm, Ø_{int.}=1.22 mm, Hilgenberg GmbH, Malsfeld, Germany) (Hamill et al., 1981). Current and voltage electrodes of a diameter of 1-3 μm are filled with 1.5 M KCl and 1.5 M potassium acetate. The pipettes have a capacitance of 0.2-0.5 MW. For the electrophysiological measurements, the oocytes are transferred into a small chamber which is flushed continuously with normal Rimland solution (in mM: KCl 90, MgCl₂ 3, HEPES 5, pH 7.2). To apply a substance, the perfusion solution is exchanged for a substance solution of the same composition which additionally comprises the desired concentration of substance. The successful expression of the receptor DNA is checked after one week at a clamp potential of −60 mV. Unresponsive oocytes are discarded. All the others are used for substance testing. The data are documented by means of a YT plotter (YT plotter, Model BD 111, Kipp & Zonen Delft BV, AM Delft, The Netherlands). The individual data are entered into Origin (evaluation software Microbial Origin, Microbial Software, Inc., Northampton, Mass. 01060-4410 USA) (Additive GmbH, Friedrichsdorf/Ts, Germany). Means, standard deviation, IC₅₀ values and IC₅₀ curves are calculated using Origin. These measurements are carried out at least in duplicate.

### REFERENCES

Birgul, N. et al. (1999). Reverse physiology in Drosophila: identification of a novel allatostatin-like neuropeptide and its cognate receptor structurally related to the mammalian somatostatin/galanin/opioid receptor family. EMBO Journal 18: 5892-5900
Blackburn, M. B. et al. (1995). The isolation and identification of three diuretic kinins from the abdominal ventral nerve cord of adult Helicoverpa zea. J. Insect Physiol. 41 (8): 723-730
Brown, B. E. und Starrall, A. N. (1975). Isolation of proctolin, a myotropic peptide from Periplaneta americana. J. Insect Physiol. 21: 1879-1881
Coast, G. M. (1998). Insect Diuretic Peptides: Structures, Evolution and Actions. American Zoology 38: 442-449
Conklin et al. (1993). Substitution of three amino acids switches receptor specificity of Gq alpha to that of Gi alpha. Nature 363:274-276
Cox, K. J. et al. (1997). Cloning, characterization, and expression of a G-protein-coupled receptor from Lymnea stagnalis and identification of a leucokinin-like peptide, PSFHSWSamide, as ist endogenous ligand. J. Neurosci. 17:1197-1205
Devereux et al. (1984). Nucleic Acids Research 12: 387
Dumont, J. N. (1972). Oogenesis in Xenopus laevis (Daudin). 1. Stages of oocyte development in laboratory maintained animals. J. Morphol. 136: 153-180
Eriksen, K. K. (2000). Molecular cloning, genomic organization, developmental regulation, and a knock-out mutant of a novel leu-rich repeats-containing G-protein-coupled receptor (DLGR-2) from Drosophila melanogaster. Genome Res. 10:924-938
ffrench-Constant, R. H. et al. (1991). Molecular cloning and transformation of cyclodiene resistance on Drosophila and invertebrate GABA_{A} receptor locus. Proc. Natl. Acad. Sci. U.S.A. 88: 7209-7213
Gäde, G. et al. (1997a). Hormonal regulation in Insects: Facts, Gaps, and Future Directions. Physiological Reviews 77: 963-1032
Gäde, G. (1997b). The explosion of structural information on insect neuropeptides, In: Progress in the chemistry of organic natural products (Herz, W., Kirby, G. W., Moore, R. E., Steglich, W., Tamm, C. eds): 1-128
Ha, S.-D. et al (2000). Cloning and sequence analysis of cDNA for Diuretic Hormone Receptor from the Bombyx mori. Mol. Cells 10: 13-17
Hamill, O. P. et al. (1981). Improved patch-clamp techniques for high-resolution current recording from cells and cell-free membrane patches. Pfügers Arch. 391: 85-100
Hauser, F. et al. (1998). Molecular cloning, genomic organization and developmental regulation of a novel receptor from Drosophila melanogaster structurally related to Gonadotropin-Releasing Hormone Receptors from vertebrates. Biochem Biophys. Res. Comm. 249: 822-828
Hay et al. (1997). P element insertion-dependent gene activation in the Drosophila eye. Proc. Natl. Acad. Sci. U.S.A 94 (10): 5195-5200
Hewes, R. und Taghert, P. (2001). Neuropeptides and neuropeptide receptors in the Drosophila melanogaster genome. Genome Research 11 (6): 1126-1142
Holman, G. M. et al. (1986). Isolation, primary structure and synthesis of two neuropeptides from Leucophaea maderae: Members of a new family of Cephalomaotrophins. Comparative Biochemical Physiology 84C: 205
Holmes, S. P. et al. (2000). Cloning and transcriptional expression of a leucokinin-like peptide receptor from the Southern cattle tick, Boophilus microplus (Acari:Ixodidae). Insect Molecular Biol. 9 (5): 457-465
Holman, G. M. (1991). Insect myotropic peptides: isolation, structural characterization and biological properties. In: Insect Neuropeptides: Chemistry, Biology and Action (Menn, J. J., Kelly, T. J., Masler, E. P., eds): 40-50
King, F. D. und Wilson, S. (1999). Recent advances in 7-transmembrane receptor research. Current Opinion in Drug Discovery & Development 2: 83-95
Lagueux, M. et al. (1990). cDNAs from neurosecretory cells of brains of Locusta migratoria encoding a novel member of the superfamily of insulins. European Journal of Biochemistry 187: 249-254
Lenz et al. (2000a). Molecular cloning and genomic organization of a novel receptor from Drosophila melanogaster structurally related to mammalian Galanin Receptors. Biochem. Biophys. Res. Comm. 269: 91-96
Lenz et al. (2000b). Molecular cloning and genomic organization of a second probable allatostatin receptor from Drosophila melanogaster. Biochem. Biophys. Res. Comm. 273: 571-577
Li, X. J. et al. (1991). Cloning, heterologous expression and developmental regulation of a Drosophila receptor for tachykinin-like peptides. EMBO J. 10: 3221-3229
Monnier, D. et al. (1992). NKD, a developmentally regulated tachykinin receptor in Drosophila. J. Biol. Chem. 267: 1298-1302
Osborne, R. H. (1996). Insect Neurotransmission: Neurotransmitters and their Receptors. Pharmacology & Therapeutics 69: 117-142
Pietrantonio, P. V. et al. (2000). Characterization of a leucokinin binding protein in Aedes aegypti (Diptera: Culicidae) Malpighina tubule. Insect Biochemistry and Molecular Biology 30: 1147-1159
Plasterk (1996). The Tcl/mariner transposon family, Transposable Elements/Current Topics in Microbiology and Immunology 204: 125-143
Ramsey, J. A. (1954). Active transport of water by the Malpighian tubules of the stick insect, Dixippus morosus. Journal of Experimental Biology 31: 104-113
Reagan, J. D. (1994). Expression cloning of an insect diuretic hormone receptor. Journal of Biological Chemistry 269: 9-12
Sambrook et al. (1989). Molecular Cloning, A Laboratory Manual, 2nd ed. Cold Spring Harbour Press
Seinsche, A. et al. (2000). Effect of helicokinins and ACE inhibitors on water balance and development of Heliothis virescens larvae. Journal of Insect Physiology 46: 1423-1431
Stables et al. (1997). A Bioluminescent Assay for Agonist Activity at Potentially Any G-protein coupled Receptor. Analytical Biochemistry 252: 115-126
Stratowa C. et al. (1995). Use of a luciferase reporter system for characterizing G-protein-linked receptors. Current Opinion in Biotechnology 6: 574-581
Terhzaz, S. et al. (1999). Isolation and characterization of a leucokinin-like peptide of Drosophila melanogaster. J. Exp. Biol. 202: 3667-3676
Tobe, S. S. et al. (1994). Allatostatins, peptide inhibitors of juvenile hormone production in insects. In: Perspectives in Comparative Endocrinology (Davey, K. G., Peter, R. E., Tobe, S. S., eds): 12-19
Torfs, P. et al. (1999). The kinin petide family in invertebrates. Ann. NY Acad. Sci. 897: 367-373
Vanden Broeck, J. et al. (1997). Insect Neuropeptides and Their Receptors. Trends in Endocrinology & Metabolism 8: 321-326
Vanden Broeck, J. (2001). Insect G protein-coupled receptors and signal transduction. Archieves of Insect Biochemistry and Physiology 48: 1-12
White J. H. et al. (1998). Heterodimerization is required for the formation of a functional GABA(B) receptor. Nature 396: 679-82.

1. Polypeptide having the biological activity of a helicokinin receptor and comprising an amino acid sequence which has at least 70% identity with the sequence of SEQ ID NO: 2

2. Polypeptide according to claim 1, characterized in that the amino acid sequence corresponds to the sequence of SEQ ID NO: 2.

3. Polynucleotide comprising a nucleotide sequence which encodes a polypeptide according to claim 1.

4. Polynucleotide according to claim 3, characterized in that it is a single-stranded or double-stranded DNA or RNA.

5. Polynucleotide according to claim 4, characterized in that it is a fragment of genomic DNA or cDNA.

6. Polynucleotide according to claim 3, characterized in that the nucleotide sequence corresponds to the sequence of SEQ ID NO: 1.

7. Polynucleotide according to claim 3, characterized in that it hybridizes under stringent conditions to the sequence of SEQ ID NO: 1.

8. DNA construct comprising a polynucleotide according to any of claims 3 to 7 and a heterologous promoter.

9. Vector comprising a polynucleotide according to any of claims 3 to 7 or a DNA construct according to claim 8.

10. Vector according to claim 9, characterized in that the polynucleotide is linked functionally to regulatory sequences which ensure the expression of the polynucleotide in pro- or eukaryotic cells.

11. Host cell containing a polynucleotide according to any of claims 3 to 7, a DNA construct according to claim 8 or a vector according to claim 9 or 10.

12. Host cell according to claim 11, characterized in that it is a prokaryotic cell, in particular E. coli.

13. Host cell according to claim 11, characterized in that it is a eukaryotic cell, in particular a mammalian or insect cell.

14. Antibody which binds specifically to a polypeptide according to claim 1.

15. Transgenic invertebrate containing a polynucleotide according to any of claim 3 to 7.

16. Transgenic invertebrate according to claim 15, characterized in that it is Drosophila melanogaster or Caenorhabditis elegans.

17. Transgenic progeny of an invertebrate according to claim 15 or 16.

18. Method of preparing a polypeptide according to claim 1, comprising
(a) culturing a host cell according to any of claims 11 to 13 under conditions which ensure the expression of the polynucleotide according to any of claims 3 to 7, or
(b) expressing a polynucleotide according to any of claims 3 to 7 in an in vitro system,
(c) obtaining the polypeptide from the cell, the culture medium or the in vitro system.

19. Method of preparing a polynucleotide according to any of claims 3 to 7, comprising the following steps:
(a) Full chemical synthesis in a manner known per se, or
(b) chemical synthesis of oligonucleotides, labelling of the oligonucleotide, hybridizing the oligonucleotides to DNA of a genomic library or cDNA library generated from insect genomic DNA or insect mRNA, respectively, selecting positive clones and isolating the hybridizing DNA from positive clones, or
(c) chemical synthesis of oligonucleotides and amplification of the target DNA by means of PCR.

20. Method of generating a transgenic invertebrate according to claim 15 or 16, which comprises introducing a polynucleotide according to any of claims 3 to 7 or a vector according to claim 9 or 10.

21. Method of finding novel active compounds for crop protection, in particular compounds which alter the properties of polypeptides according to claim 1, comprising the following steps:
(a) Providing a host cell according to any of claims 11 to 13,
(b) culturing the host cell in the presence of a chemical compound or a sample which comprises a multitude of chemical compounds,
(c) detecting altered properties.

22. Method of finding a chemical compound which binds to a polypeptide according to claim 1, comprising the following steps:
(a) Bringing a polypeptide according to claim 1 or a host cell according to any of claims 11 to 13 into contact with a chemical compound or a mixture of chemical compounds under conditions which permit the interaction of a chemical compound with the polypeptide, and
(b) determining the chemical compound which binds specifically to the polypeptide.

23. Method of finding a chemical compound which alters the expression of a polypeptide according to claim 1, comprising the following steps:
(a) Bringing a host cell according to any of claims 11 to 13 or a transgenic invertebrate according to claim 15 or 16 into contact with a chemical compound or a mixture of chemical compounds,
(b) determining the concentration of the polypeptide according to claim 1, and
(c) determining the chemical compound which specifically affects the expression of the polypeptide.

24. Use of a polypeptide according to claim 1, of a polynucleotide according to any of claims 3 to 7, of a vector according to claim 9 or 10, of a host cell according to any of claims 11 to 13, of an antibody according to claim 14, or of a transgenic invertebrate according to claim 15 or 16 for finding novel active compounds for crop protection or finding genes which encode polypeptides which participate in the synthesis of functionally similar helicokinin receptors in insects.

25. Use of a modulator of a polypeptide according to claim 1 as insecticide.


SEQUENCE LISTING (2 sequences)

SEQ ID NO: 1 - DNA, 1452 bp, Heliothis virescens
  CDS (1)..(1452)
atggacactagtacaacaaattcatcacaagatgacgacgcggactggccaaggaacagttccattgacgagtatattatacacaatggaactaatgatacattcgaaacattgtacgatgtgccgactggtatgatagtactcttgtcgttcctgtacggctcaatatcagttcttgcggtggtggggaactttctggtgatgtgggtcgtggccacctcgagaagaatgcagagcgtcacaaactgctacatagccaacttagctttagctgacatagtcataggattattcgctgtaccatttcaatttcaagccgcgctgctacagcggtggctgctaccgcacttcatgtgtccgttctgcccgttcgtgcaggcgctcagtgtcaacgtcagcgtgtttacactgacagccatcgcagttgacagacatcgggcgataatcacaccgctcagcgcccacacttcaaagcgtattgccaaagtaataatagtggttatatggtttctggcgctttctttagctgctccgatggctatgtcttgggaggttatcatggaagatgaattagatccagttgcaaaaatcttctacaaaaagccgttttgtgcacccaccgagttcggctcgcattcactcgccatttatagactgttgttgtatgtatttcagtatgtaatcccgttgtgtgtgattacgtttgcctacgctcatatggcgatgaagctgtggggagcgcgcgccccagggaacgcgcaggagacgagggacgctaaccacatgcgaaacaagaagaaggtgataaaaatgttggtgctggtcgtagctctgtttgcgttatgctggctgccgctacagagctacttattactacaatcattttttccatcaattaacgagtacaagtacatcaacgtgcttttcttttgcttcgactggctagcaatgagcaactcttgctataacccattcatctatgccatctacaacgaaaaattcaagaaggaattcaaacaacgattcactttcgggaaaaagccaagcagattcgttaacgatagctacgaggacggccagtcataccgaacaagaattttatcgttccgatcaaccaacgacagaagtggctattcatccagaaagtctttgaacataccgccgggggacactttaaaagttccttctagaaattcatgtcattgcatggcgaatcagagcagagaaaatggatttaacttcatgaaaactgaagacatggaagggcacggaaatagcaggcggtatctgaatataagaatgagtaatccagatattggtaaaagaagattagctaagaagttatcgaatagagacgacatgcctataggtgatgagagagtcagtgaactgtacatattcccaaacagtaacattgtagaatttacagacatatcatacgatgacaaagtg

SEQ ID NO: 2 - protein, 484 aa, Heliothis virescens
MDTSTTNSSQDDDADWPRNSSIDEYIIHNGTNDTFETLYDVPTGMIVLLSFLYGSISVLAVVGNFLVMWVVATSRRMQSVTNCYIANLALADIVIGLFAVPFQFQAALLQRWLLPHFMCPFCPFVQALSVNVSVFTLTAIAVDRHRAIITPLSAHTSKRIAKVIIVVIWFLALSLAAPMAMSWEVIMEDELDPVAKIFYKKPFCAPTEFGSHSLAIYRLLLYVFQYVIPLCVITFAYAHMAMKLWGARAPGNAQETRDANHMRNKKKVIKMLVLVVALFALCWLPLQSYLLLQSFFPSINEYKYINVLFFCFDWLAMSNSCYNPFIYAIYNEKFKKEFKQRFTFGKKPSRFVNDSYEDGQSYRTRILSFRSTNDRSGYSSRKSLNIPPGDTLKVPSRNSCHCMANQSRENGFNFMKTEDMEGHGNSRRYLNIRMSNPDIGKRRLAKKLSNRDDMPIGDERVSELYIFPNSNIVEFTDISYDDKV